(19) United States
(12) Patent Application Publication
(10) Pub. No.: US 2015/0099944 A1
(43) Pub. Date: Apr. 9, 2015

(54) Medical monitoring tablet and related smartphone application

(71) Applicant: Brandon James, Detroit, MI (US)

(72) Inventor: Brandon James, Detroit, MI (US)

(21) Appl. No.: 14/508,622

(22) Filed: Oct. 7, 2014

Related U.S. Application Data
(60) Provisional application No. 61/887,925, filed on Oct. 7, 2013.

(51) Int. Cl. A61B 5/00; G01N 27/30; A61B 7/04; A61B 5/0205; A61B 5/145

(52) U.S. Cl. CPC A61B 5/6898; A61B 5/02055; A61B 5/0022; A61B 5/14542; A61B 7/04; G01N 27/307; A61B 5/021
USPC 600/301

(57) ABSTRACT

A medical monitoring tablet and related application for a smartphone features a testing strip port for blood glucose testing strips used in diabetes monitoring, as well as USB ports for the transfer of monitoring data, and may feature one or more ports for other electronic monitoring devices such as a stethoscope, a blood pressure cuff, and a thermometer. A related smartphone application provides visual and audible reminders to the user when blood glucose, blood pressure, or other medical data needs to be gathered, and medication needs to be taken. The tablet also features a front side with a touchscreen for viewing the test results, a keyboard display on the touchscreen to command the tablet to perform different functions. Once the medical function is performed, it is transferred by the tablet, and then displayed on the touchscreen or on the smartphone application.

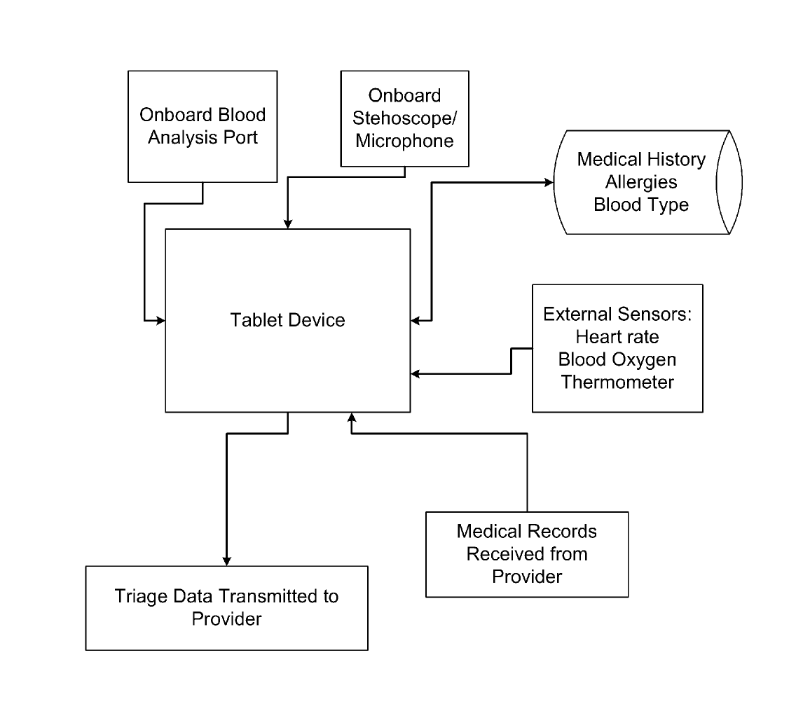

### CROSS-REFERENCE TO RELATED APPLICATIONS

This application claims the benefit of U.S. Provisional Application No. 61/887,925, filed on Oct. 7, 2014, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM

LISTING COMPACT DISK APPENDIX

Not Applicable

### BACKGROUND OF THE INVENTION

The invention relates generally to medical monitoring devices, and in particular to a medical monitoring tablet and a related application for a smartphone. For many medical patients, following up on a long-term medical condition with self-care at home can present a challenge. Many diabetics, heart disease patients, and others with chronic illnesses which are treatable fail to control their illnesses with the proper monitoring and medications, resulting in further deterioration of their conditions, and even death. If the monitoring of their conditions could be consolidated into a single device and transmitted to their physician via a smartphone application, it would make monitoring easier and more convenient, helping to resolve the serious problem of diagnosed, but uncontrolled chronic medical conditions.

### SUMMARY OF THE INVENTION

Accordingly, the invention is directed to a medical monitoring tablet and related application for a smartphone. A tablet features a testing strip port for blood glucose testing strips used in diabetes monitoring, as well as Universal Service Bus (USB) ports for the transfer of monitoring data, and may feature one or more ports for other electronic monitoring devices. The tablet also features a built-in stethoscope for the monitoring of cardiac rate, and a built-in thermometer to gather body temperature data. A related smartphone application provides visual and audible reminders to the user when blood glucose, blood pressure, or other medical data needs to be gathered, and medication needs to be taken. The tablet also features a front side with a touchscreen for viewing the test results, a keyboard display on the touchscreen to command the tablet to perform different functions, a microchip, one or more USB ports, and a battery compartment. The testing strips are disposable and made for only one use. A lancet is used to collect the blood sample from the patient and transfer it onto the testing strip. Once the testing strip has been inserted into the device, the blood glucose data can be measured, calculated, and transferred by the tablet, and then displayed on the touchscreen or on the smartphone application. The tablet may also be used conventionally for Internet browsing.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWING

The accompanying drawing is included to provide a further understanding of the invention and is incorporated into and constitutes a part of the specification. It illustrates one embodiment of the invention and, together with the description, serves to explain the principles of the invention.

The FIGURE is a flowchart displaying the system components and processes and their interconnections, and other relevant events during the course of using the invention.

### DETAILED DESCRIPTION OF THE INVENTION

Referring now to the invention in more detail, the invention is directed to a medical monitoring tablet and a related application for a smartphone. For many medical patients, following up on a long-term medical condition with self-care at home can present a challenge. Many diabetics, heart disease patients, and others with chronic illnesses which are treatable fail to control their illnesses with the proper monitoring and medications, resulting in further deterioration of their conditions, and even death. If the monitoring of their conditions could be consolidated into a single device and transmitted to their physician via a smartphone application, it would make monitoring easier and more convenient, helping to resolve the serious problem of diagnosed, but uncontrolled chronic medical conditions. A tablet features a testing strip port for blood glucose testing strips used in diabetes monitoring, as well as Universal Service Bus (USB) ports for the transfer of monitoring data, and may feature one or more ports for other electronic monitoring devices such as a stethoscope, a blood pressure cuff, and a thermometer. A related smartphone application provides visual and audible reminders to the user when blood glucose, blood pressure, or other medical data needs to be gathered, and medication needs to be taken. The tablet also features a front side with a touchscreen for viewing the test results, a keyboard display on the touchscreen to command the tablet to perform different functions, a microchip, one or more USB ports, and a battery compartment. The testing strips are disposable and made for only one use. A lancet is used to collect the blood sample from the patient and transfer it onto the testing strip. Once the medical function is performed, it is transferred by the tablet, and then displayed on the touchscreen or on the smartphone application. The tablet may also be used conventionally for Internet browsing.

Electronic components of the tablet would preferably include a touchscreen, an on/off button or switch, a navigation button to navigate between one monitoring function and the next, a microprocessor, software necessary to operate the device, one or more USB ports, a stethoscope and thermometer contained in cavities in the body of the device and connected to the device via retracting cables, a testing strip port, a transmitter, a receiver, and a battery contained in the battery compartment, all contained within the body. The battery would preferably be of a standard, off-the-shelf type, commonly used in smartphones or tablet computers. The body would preferably be manufactured from durable, rigid materials such as steel, aluminum and high impact plastic, and would preferably be four inches long, two and one-half inches wide, and one-half inch thick.

The first exemplary embodiment of the device would preferably be enabled to transmit the results of a blood glucose test, body temperature, and other medical monitoring functions to a computer or smartphone via the USB port, or via any one of several wireless data communication protocols, such as WiFi™ or Bluetooth™.

Accessories provided with the tablet, or separately, may include a cardiac rate monitor, a sphygmomanometer with blood pressure cuff, and other medical monitoring devices. Blood oxygen may be monitored by a sensor contained within the tablet, or via testing strips inserted into the testing strip port. All accessories are powered by batteries, standard household current, or both, and are capable of transferring medical monitoring data to the tablet via a USB cable which may be connected to a USB port on the tablet.

The smartphone application and the software necessary to operate the tablet collect and store the various types of medical monitoring data, as well as the user's medical history from health care providers, and either store it for retrieval by the user, or make it available in encrypted form for access by the user's health care providers, including paramedics or other first responders in the event of a medical emergency. The software application and the software record in a local database the blood type, Rh-factor, and any allergies the user may have, and may also provide comparison and charting functions, such as comparing the medical monitoring data to normal ranges for the measured data. The software application and the software are capable of recording and performing comparison and charting functions for one user, or for any or all persons in the user's household, according to a plurality of distinct user profiles stored within said local database.

To use the tablet and smartphone application, the user or the user's health care provider transfers the blood type, Rh-factor, and any allergy information and other medical history data to the tablet using a USB port, the keyboard display on the touchscreen, or both. Medical monitoring functions and medications, as well as the time or times each day for medical monitoring functions and the taking of the medications, are also transferred to the tablet. The smartphone application is used to copy this data, using either a USB cable and USB port, or an encrypted data transmission using the selected wireless data transmission protocol. The user also selects the reminders to be used on the smartphone, which may include different reminders such as ringtones, text messages, or visual icons, for the different medical monitoring functions, medications, and members of the user's household. Such reminders are stored and retrieved according to a schedule within the local database.

When the user is prompted via audible reminders, visual reminders, or both on the smartphone, the user performs the required medical monitoring functions, takes the required medications, or administers these to a household member as prompted. This may involve taking a blood droplet with a lancet and placing it on a testing strip, then inserting the testing strip in the testing strip port; using the built-in thermometer to take the body temperature; using the built-in electronic stethoscope to measure cardiac rate; using accessories, such as a sphygmomanometer with blood pressure cuff, to monitor other physiological functions and conditions; or some combination of these activities. As it is gathered, medical monitoring data may be stored in the local database and periodically transferred to the smartphone, using a USB port or an encrypted data transmission. The user may then review the gathered medical monitoring data, using the comparison and charting functions of the software and the smartphone application as desired, and transfer the gathered medical monitoring data from the tablet or smartphone to one or more health care providers, using a USB port or an encrypted data transmission.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A medical monitoring tablet comprising a tablet device and a software application method; said software application method being operated by said tablet device; said tablet device being equipped with a testing strip port; said testing strip port being in electronic communication with said tablet device; said software application method having the capability to read information from blood glucose test strips inserted into said test strip port; said software application method being configured to store to and retrieve from a local database; a temperature sensor; said temperature sensor being in electronic communication with said tablet device; said software application method having the capability to read information from said temperature sensor; a blood oxygen sensor; said blood oxygen sensor being in electronic communication with said tablet device; said software application method having the capability to read information from said blood oxygen sensor; an electronic stethoscope; said electronic stethoscope being in electronic communication with said tablet device; said software application method having the capability to read information from said electronic stethoscope; an electronic sphygmomanometer;
said electronic sphygmomanometer being in electronic communication with said tablet device; said software application method having the capability to read information from said electronic sphygmomanometer; said local database containing data pertaining to a user comprising any of the group of allergies, blood type, Rh-factor, schedule of medications, schedule of medical monitoring functions, or past monitoring data; said software application method being configured to present any of the group of visual reminders or audible reminders according to said schedule of medications or to said schedule of medical monitoring functions; said software application method being configured to display data in said local database as a medical chart pertaining said user; said software application method being configured to export data from said local database.

2. The electronic tablet device of claim 1 wherein exporting data from said local database comprises transmitting data to a smartphone operated by said user.

3. The electronic tablet device of claim 1 wherein exporting data from said local database comprises transmitting data to one or more health care providers.

4. The electronic tablet device of claim 1 wherein said software application method is capable of maintaining distinct user profiles within said local database for a plurality of users.

5. The electronic tablet device of claim 2 wherein said software application method is capable of maintaining distinct user profiles within said local database for a plurality of users.

6. The electronic tablet device of claim 3 wherein said software application method is capable of maintaining distinct user profiles within said local database for a plurality of users.

7. The electronic tablet device of claim 2 wherein exporting data from said local database comprises transmitting data to one or more health care providers.

8. The electronic tablet device of claim 5 wherein exporting data from said local database comprises transmitting data to one or more health care providers.
